SEQUENCE LISTING (11 sequences)

SEQ ID NO: 1 - protein, 839 aa, Bacillus subtilis
MKKRGVSNMYSVLFRQAEESSQLAGAKGMNLIKLTKHGLPVPDGFIIQTNALARFMEDNQLQETSENVESGIISGTFSDELKDELTSSFYKLRESYRSVAVRSSSASEDLEGASFAGQYETYLNIKTEEEFLAKVKECWASFFSGRVSSYKKKMNNQIAEPLMGIVVQGLIDSEMSGVIFSRNPVTHDDRELLISASYGLGEAVVSGSVTPDTFIVNKSSFEIQKEIGAKEIYMESAAEGIAEKETSEDMRSRFCLTDEQVIELAEITKKTEDLYGYPVDIEFGIADHQIYLLQARPITTIDQDKKAAEEKRSFMITDTDMNDFWLNMESNIEGPVSPLFSSFIVPALEYGLKKSMQKFPIGVVVDEVKLYRGHIYSKNQGGQQPPSEDCGKELFPILSEHMYDIINHTYLPFYRTLDQLAQTEHTAESALDAFQKLKAFYLTAYEEHFNIVFPQILLTNKLQAMYQDIQGESENAHFYEMLTGKMNKSLETDRCLWLFSMEVQENPNLLTIFENNKPEQLQEKLEQTDEGRHFLKNVHEFLQEYGWRSVKSHDLIEQIWVENPYFALANIQNYVRNGYHFDNEFQKTKEKREKLYNEFLENIEDPGLRTEFDRYYQWTLNSANIKDDHHFYIDAMLDAKARIFLLKIGELLAENGVIQDREDLWFLYDDEVEQALLHPVSLQEKAEKRRQIFHEYELAQAPAYLGTPTKEQLKAAEEIVGAVIEDEKNTENHIFGIAASSGIATGPVKIIRDANEFSQFAPGDVLVCKMTTPLWTSLFQDAKAIITDTGGILSHAAIIAREYGIPAVLGTRTATERLRDGDIITVDGSSGKITVVSRS

SEQ ID NO: 2 - protein, 16 aa, synthetic construct
  source 1..16: note = conserved motif
  REGION 1..16
DDHHFYIDAMLDAKAR

SEQ ID NO: 3 - protein, 289 aa, Bacillus subtilis
AGAKGMNLIKLTKHGLPVPDGFIIQTNALARFMEDNQLQETSENVESGIISGTFSDELKDELTSSFYKLRESYRSVAVRSSSASEDLEGASFAGQYETYLNIKTEEEFLAKVKECWASFFSGRVSSYKKKMNNQIAEPLMGIVVQGLIDSEMSGVIFSRNPVTHDDRELLISASYGLGEAVVSGSVTPDTFIVNKSSFEIQKEIGAKEIYMESAAEGIAEKETSEDMRSRFCLTDEQVIELAEITKKTEDLYGYPVDIEFGIADHQIYLLQARPITTIDQDKKAAEEKR

SEQ ID NO: 4 - protein, 70 aa, Bacillus subtilis
PGDVLVCKMTTPLWTSLFQDAKAIITDTGGILSHAAIIAREYGIPAVLGTRTATERLRDGDIITVDGSSG

SEQ ID NO: 5 - DNA, 1347 bp, Bacillus subtilis
  source 1..1347: mol_type = other DNA
agcttcatgattaccgacactgatatgaatgatttctggcttaacatggagtctaatattgaaggtccggtgagtccgttattttcatccttcatcgtgccggcattggaatatggcttgaagaagagcatgcaaaagtttccgattggtgtagttgttgatgaagtaaaactttatcgcggacatatttattccaaaaaccaaggtggacagcagcctccttctgaagactgcggcaaagagcttttcccgattttatcggagcatatgtatgacatcatcaatcacacatacctccctttttaccggacactggaccagctcgcacaaactgagcataccgcagaaagcgcactggatgcttttcaaaaactaaaggccttttatctcacggcttatgaagagcacttcaatatcgttttcccgcaaatccttttaacaaacaaactgcaagcgatgtatcaggacattcaaggagagtccgaaaacgctcatttttatgagatgctgacaggaaaaatgaacaaatcactggaaacggaccgttgcttatggctattttctatggaagttcaggagaacccgaaccttctgaccatttttgaaaacaacaagcctgaacagctccaggagaaattagaacaaacagatgaggggagacacttcctgaagaacgtccatgaattcttgcaagaatacggatggagatctgttaaaagtcatgatctgattgaacaaatctgggtggaaaatccgtatttcgctctggctaatattcaaaattatgtccgtaatggctatcattttgacaatgaatttcagaaaacgaaagaaaaacgagagaaattatacaatgaattcttggaaaacatagaagatcccggtttgcgcaccgaatttgaccgctattatcaatggacactgaactctgcaaatataaaagatgatcaccacttttatattgacgccatgctggatgccaaggcgagaatctttctgctgaagataggtgaattgctggcggaaaacggtgtcattcaagatcgtgaggacctttggtttttatatgacgacgaagtggaacaagcgcttcttcaccctgtatccctgcaagaaaaggctgaaaaacgcagacagatttttcatgagtatgagctggcccaagcccctgcctacctcggcaccccgacaaaagaacagctcaaagcagctgaagaaattgtcggcgctgtgatagaggatgaaaaaaacacagagaatcatatttttggcattgcggcatcaagcggcattgcgacaggtccggtgaaaatcattcgggacgccaatgaattttctcaattcgcg

SEQ ID NO: 6 - DNA, 936 bp, Bacillus subtilis
  source 1..936: mol_type = other DNA
atgaagaaaagaggggtttcaaatatgtattctgttttatttcgccaggcagaagagtccagccagctggctggagcaaaaggaatgaatttgattaaattgaccaaacacggtcttcctgttccggacgggtttattattcaaacgaatgcgctcgcacgttttatggaggacaaccagcttcaagagactagtgaaaacgtcgaaagcgggatcatttctggaacattttcggatgagctgaaagatgagctgactagttccttttataagcttagagaatcatatcgatccgtagccgtgcgttcttcgtctgcttcggaagatttagaaggcgcctcattcgcgggtcaatatgaaacctacttaaatatcaaaacagaggaagagtttctggctaaagtgaaagaatgctgggcctcatttttttctgggcgggtcagcagctataagaaaaaaatgaacaatcaaatcgcagagccgttaatgggaatagtcgttcaggggctgatcgattcagaaatgtcaggtgttatcttcagccgcaaccctgttacccatgatgatagagagcttttaatcagcgccagctacgggttgggtgaagctgttgtttcaggaagtgttaccccagacacgttcattgttaataaatcttcgtttgagattcagaaagaaataggtgcaaaggaaatctacatggagtctgcggcagaaggaattgctgaaaaagaaacgagtgaagacatgcgcagccgtttttgccttacagatgaacaagtgattgaattggctgaaatcacaaaaaaaaccgaagacctgtacggatatcctgtcgatatagaatttggaattgctgatcatcaaatataccttctgcaagctcgcccgattacaaccattgatcaggacaaaaaggcggcagaagaaaaacgc

SEQ ID NO: 7 - DNA, 237 bp, Bacillus subtilis
  source 1..237: mol_type = other DNA
cctggggacgtactcgtttgcaagatgaccacaccgctatggaccagcctgtttcaagacgccaaagcgataattacagacacaggcggcattttgtctcacgctgcgattattgcccgtgaatacggcattccagccgttctcggcacacgcacggcaaccgaaagactgcgagacggtgacatcatcactgttgacggtagcagcggcaaaatcacagttgtcagccggtcctga

SEQ ID NO: 8 - protein, 286 aa, Arthrobacter aurescens
MPMVAAVEFAKSPAEVLRVGSGFSLAGVDPESTPGYTGVKADGKALLAAQDARLAELQEKLFAEGKFGNPKRLLLILQAMDTAGKGGIVSHVVGAMDPQGVQLTAFKAPTDEEKSHDFLWRIEKQVPAAGMVGVFDRSQYEDVLIHRVHGWADAAELERRYAAINDFESRLTEQGTTIVKVMLNISKDEQKKRLIARLDDPSKHWKYSRGDLAERAYWDDYMDAYSVAFEKTSTEIAPWHVVPANKKWYARIAVQQLLLDALGGLQLDWPKADFDVAAERALVVES

SEQ ID NO: 9 - protein, 277 aa, Deinococcus proteolyticus
MNSKASQQLQVPPGQKVRLADYSTDTFEGSAALDKEQVKQATEQLQQRLSELQEKLYAEGKQSLLIILQARDGGGKDSTVSRVMGAFNPNGVHVANFKAPTDLELQHDFLWRIHQQVPSHGMIAVFNRSHYEDVLVTRVHGLIDDARAQQNLEHIVNFEKLLSDAGTRIVKFYLHLSPEEQKARMEDRLNDPAKHWKFNPSDLKDRALWHEYTAAYEDALATSRSYAPWYIIPADRKWLRDYLISEILVQTLEEMNPQFPTAHFEAAYYLIEDIPSR

SEQ ID NO: 10 - protein, 305 aa, Cytophaga hutchinsonii
MATDFSKLSKYVETLRVKPKQSIDLKKDFDTDYDHKMLTKEEGEELLNLGISKLSEIQEKLYASGTKSVLIVFQAMDAAGKDGTVKHIMTGLNPQGVKVTSFKVPSKIELSHDYLWRHYVALPATGEIGIFNRSHYENVLVTRVHPEYLLSEQTSGVTAIEQVNQKFWDKRFQQINNFEQHISENGTIVLKFFLHVSKKEQKKRFIERIELDTKNWKFSTGDLKERAHWKDYRNAYEDMLANTSTKQAPWFVIPADDKWFTRLLIAEIICTELEKLNLTFPTVSLEQKAELEKAKAELVAEKSSD

SEQ ID NO: 11 - protein, 298 aa, unidentified
  source 1..298: note = Erysipelotrichaceae
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEK

(19) United States
(12) Patent Application Publication
(10) Pub. No.: US 2024/0318212 A1
(43) Pub. Date: Sep. 26, 2024

(54) PHOSPHORYLATION OF PHENOLIC PHYTOCHEMICALS BY TWO ENZYMES COUPLED SYSTEM

(71) Applicant: National Taiwan University, Taipei (TW)

(72) Inventors: Nan-Wei SU, Taipei (TW); Hsin-Ya TSAI, Taipei (TW); Chen HSU, Taipei (TW)

(21) Appl. No.: 18/599,745

(22) Filed: Mar. 8, 2024

Related U.S. Application Data
(60) Provisional application No. 63/451,238, filed on Mar. 10, 2023.

(51) Int. Cl. C12P 17/18; C12N 9/00; C12N 9/12; C12P 17/06

(52) U.S. Cl. CPC C12P 17/181; C12N 9/1229; C12N 9/93; C12P 17/06; C12Y 207/04001; C12Y 602/01005

(57) ABSTRACT

The present invention provides a coupled enzyme system, comprises: a first enzyme, comprising a polyphenol phosphorylation synthetase; a second enzyme, which is ATP regeneration enzyme; and a substrate, being phosphorylated by the first enzyme. The coupled enzyme system of the present invention integrates polyphenol phosphorylation synthetase with ATP regeneration enzyme so that the polyphenol phosphorylation synthetase is used to phosphorylate polyphenol and the ATP regeneration enzyme regenerate ATP from AMP. Therefore, the present invention not only improves the water-solubility and bioavailability of the phenolic phytochemicals but also significantly reduces ATP consumption, presenting the potential of enzymatic systems in the production of polyphenol monophosphates.

### CROSS REFERENCE TO RELATED APPLICATIONS

This application claims priority of U.S. Provisional Application No. 63/451,238 filed on Mar. 10, 2023 under 35 U.S.C. § 119(e), the entire contents of all of which are hereby incorporated by reference.

### REFERENCE TO AN ELECTRONIC SEQUENCE LISTING

The content of the electronic sequence listing (2024-06-14-SequenceListing; Size: 15,973 bytes; and Date of Creation: Jun. 14, 2024) is herein incorporated by reference in its entirety.

### FIELD OF THE INVENTION

The present invention relates to a system for producing polyphenolic phytochemicals phosphate derivative, and more particularly, a coupled enzyme system with two enzymes to phosphorylate polyphenol while reduce ATP consumption. The present invention focuses on improving the solubility and bioavailability of polyphenol and addressing the ATP requirement of phosphotransferase.

### BACKGROUND OF THE INVENTION

Phenolic phytochemicals, such as flavonoids, are abundant in plants and play a crucial role in dietary supplements and pharmaceuticals due to their diverse structures and physiological activities. The biodiversity of ecosystems offers a renewable source for discovering polyphenols with unique bioactivities. Over the past two decades, the therapeutic potential of polyphenol extracts has attracted significant attention, as indicated by over 750 clinical trials. This includes flavonoids such as luteolin (LUT), which shows promise in treating neuroinflammation-related diseases, including post-COVID-19 chronic olfactory dysfunction and autism spectrum disorders (ASD). Silybins from milk thistle are recognized for their hepaprotective properties, with ongoing investigations into their new bioactivities. Genistein, a phytoestrogen, has recently been identified as a cannabinoid receptor 1 antagonist, attenuating cannabinoid-induced atherosclerosis. However, their low aqueous solubility leads to limited bioavailability and reduced efficacy in vivo, posing a significant obstacle, as evidenced by the ineffectiveness of orally administered genistein in mitigating postmenopausal bone loss and other diseases.

To overcome the solubility issues, phosphate prodrugs have been developed to enhance the aqueous solubility and oral administration efficiency of poorly water-soluble drugs by introducing a phosphate group. This approach relies on the hydrolysis of the phosphate prodrugs into their parent drugs by endogenous alkaline phosphatase, facilitating their intestinal absorption and allowing them to exert their pharmacological effects. Monophosphates, as preferred substrates for alkaline phosphatase, constitute a group of FDA-approved phosphate prodrugs, including hydrocortisone phosphate, fosphenytoins, fosamprenavir, fluconazole, and combrestatin A-4 phosphate as a polyphenol clinical candidate. While chemical synthesis is the predominant production method, it requires complex protection and deprotection steps for the multiple hydroxy groups to minimize side products for synthesis of monophosphate esters. Enzymatic synthesis offers a greener and more efficient alternative, characterized by fewer synthetic steps and reduced waste. However, its adoption remains limited due to the scarcity of suitable biocatalysts for phospho-modification.

Previously, a novel polyphenol phosphorylation synthetase was unveiled, substantially enhancing the aqueous solubility of polyphenolic phytochemicals (U.S. Pat. No. 10,421,768 B2). Genistein 7-O-phosphate, for example, showed improved solubility and oral bioavailability, aligning with the phosphate prodrug concept. Moreover, it demonstrated improved osteoprotective effects in ovariectomized rats, even comparable to estradiol and raloxifene, medications used to treat osteoporosis, and surpassed its parent form, genistein (U.S. Pat. No. 10,881,634 B2).

### SUMMARY OF THE INVENTION

The summary of the invention aims to provide a simplified summary of the disclosure, so that the reader has a basic understanding of the disclosure. This summary of the invention is not a complete overview of the disclosure, and it is not intended to point out important/critical elements of embodiments of the invention or define the scope of the invention.

The practical application of polyphenol phosphorylation synthetase, however, is constrained by the lack of a partner enzyme for ATP regeneration, due to the ATP-dependent nature of polyphenol phosphorylation synthetase. The requirement for stoichiometric amounts of ATP, an expensive phosphate donor, makes the process economically demanding. Besides, the low aqueous solubility of polyphenolic phytochemicals, potentially hindering their accessibility to enzyme active sites, further impedes the development of an enzymatic conversion system. Addressing this issue often involves the use of cosolvents and surfactants to enhance solubility; however, these agents can destabilize enzymes, introducing additional complexity into the catalytic process.

In view of the above-mentioned issue, the present invention provides a coupled enzyme system that includes two enzymes, one is phosphotransferase, and the other is a partner enzyme for ATP regeneration; therefore, the present invention not only improves the water-solubility and bioavailability of the phenolic phytochemicals but also reduces ATP requirement.

The primary objective of the present invention is to provide a coupled enzyme system, comprises: a first enzyme, which is polyphenol phosphorylation synthetase; a second enzyme, which is ATP regeneration enzyme; and a substrate, being phosphorylated by the first enzyme.

In one aspect of the present invention, the first enzyme is an isolated or engineered polypeptide comprising a homologous protein sequence that is more than 70% identical to SEQ ID NO: 1); wherein said polypeptide sequentially comprises: an ATP-binding domain, comprising active catalytic sites of Lys27, Arg102, and Glu282; a substrate-binding domain, comprising a conserved motif of DDHIFYIDAMLDAKAR (SEQ ID NO: 2), and comprising active catalytic sites of Asp627, His629, and His630; and a phosphorylated histidine catalytic domain, comprising His795.

In one aspect of the present invention, the second enzyme is a polypeptide having a sequence that is more than 65% identical to polyphosphate Kinase 2 Class III (PPK2-III), AaPPK from A. aurescens, DpPPK from D. proteolyticus, ChPPK from C. hutchisonii, or ErPPK from an unclassified Erysipelotrichaceae bacterium; wherein said polypeptide sequentially comprises: a conserved walker A (GXDXXGK) motif of AXDXXGK for coordinating the divalent cation and polyphosphate; a conserved walker B (DR) motif of NR or DR for coordinating the nucleotide phosphate groups; active catalytic sites of Asp66, Lys70, Arg192, and Asp206 in_ErPPK (SEQ ID NO: 11) and equivalent residues.

In one aspect of the present invention, the substrate is selected from the group consisting of the following formulas:

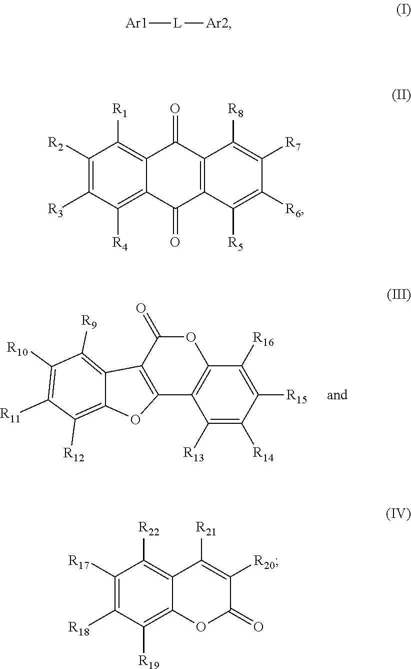

wherein Ar1 is an aryl group of the following formula:

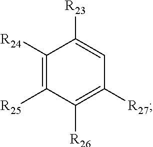

Ar2 is an aryl group of the following formula:

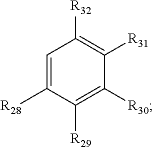

L is a linking group comprising 3 to 7 backbone carbon atoms forming a chain linking Ar1 and Ar2 as the case may be, wherein L comprises at least one of a double bond, a carbonyl group and a hydroxyl group;
R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;
R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁)phenyl group or (C₇-C₁₂)benzyl group, wherein q is an integral of 1 to 3, and at least one of R₉ to R₁₆ is a hydroxyl group;
R₁₇ to R₂₂ are respectively H, methoxy group or hydroxyl group, and at least one of R₁₇ to R₂₂ is a hydroxyl group, or R₂₀ and R₂₁, R₁₇ and R₁₈, R₁₇ and R₂₂, R₁₈ and R₁₉ or their combination are fused to form a (C₃-C₆)cycloalkyl group with hydroxyl group or a (C₆-C₁₀)aryl group with hydroxyl group;
R₂₃ to R₂₇ are respectively H, methoxy group or hydroxyl group, and at least one of R₂₃ to R₂₇ is a hydroxyl group;
R₂₈ to R₃₂ are respectively H, methoxy group or hydroxyl group, and at least one of R₂₈ to R₃₂ is a hydroxyl group;
R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₅ is (C₁-C₅)alkyl group, (C₁-C₅)haloalkoxy group, (C₆-C₁₁)phenyl group or (C₇-C₁₂)benzyl group;
R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₇ to R₄₀ are respectively (C₁-C₅)alkyl group or (C₁-C₅)haloalkoxy group;
R₄₁ and R₄₂ are respectively H, (C₁-C₅)alkyl group or (C₁-C₅)haloalkoxy group, one of which is H and the other is not H;
R₄₃ and R₄₄ are respectively H, (C₁-C₅)alkyl group or (C₁-C₅)haloalkoxy group.

In one aspect of the present invention, the reaction temperature of the system is 35 to 40° C.

In one aspect of the present invention, the reaction pH value of the system is 7.0 to 8.0.

In one aspect of the present invention, further comprises a divalent ion and a polyphosphate (polyP).

In one aspect of the present invention, the divalent metal ion is Mg²⁺ or Mn²⁺ In one aspect of the present invention, Mg²⁺ to polyP ratio is 1:1.

In one aspect of the present invention, further comprise a cosolvent and a surfactant.

In one aspect of the present invention, the surfactant is TWEEN 20 or TWEEN 80.

After referring to the following embodiments, those with ordinary knowledge in the technical field to which the present invention pertains to can easily understand the basic spirit of the present invention and its purpose, as well as the technical means and implementation aspects adopted by the present invention.

### BRIEF DESCRIPTION OF THE DRAWINGS

In order to make the above and other objects, features, advantages and embodiments of the present invention more obvious and understandable, the drawings are described as follows:

FIG. 1 illustrates the temperature dependence of PPK2-III candidates, AaPPK (a), DpPPK (b), ChPPK (c), and ErPPK (d), for catalyzing polyP-dependent phosphorylation of AMP to ADP or ATP. The reaction mixtures containing purified PPK2-III, 10 mM AMP, 20 mM MgCl₂, 20 mM polyP₆ were incubated at 30-60° C. in 50 mM Tris-HCl buffer (pH 7.5, 100 mM NaCl) for 5 min. Enzyme activity was defined as the enzyme amount required to generate 1 μmol ADP or ATP per minute, with protein measured by Bradford assay for calculating specific activity (U/mg).

FIG. 2 illustrates the cofactor (a) and polyP (b) preferences of ErPPK. Data are mean±SD (n=3). Values with different letters were significantly different by one-way ANOVA and Turkey's multiple comparison test (p<0.05). n.d., non-detectable activity.

FIG. 3 illustrates the biochemical characterization of ErPPK including temperature dependence (a), pH dependence (b), heat stability (c), and pH stability (d).

The properties of ErPPK were compared with those of BsPPS, with data for BsPPS referred to Hsu et al.

FIG. 4 illustrates the effect of polyphenol derivatives on ErPPK. Data are mean SD (n=3), with significance indicated by p<0.05 using t-test. n.s., non-significance.

FIG. 5 illustrates the effect of Mg²⁺ and polyP₆ ratio on ErPPK and BsPPS. The reaction mixtures containing varying Mg²⁺ and polyP₆ amounts with purified ErPPK and purified BsPPS were incubated at 50° C., pH 7.5 and 40° C., pH 7.8, respectively, in 50 mM Tris-HCl buffer containing 100 mM NaCl for 5 min. Enzyme activity was defined as the enzyme amount required to generate 1 μmol ATP (ErPPK) or LUT-P (BsPPS) per minute, with protein measured by Bradford assay for calculating specific activity (U/mg). Data are presented as mean±SD (n=3). Values with different letters were significantly different by one-way ANOVA and Turkey's multiple comparison test (p<0.05). n.d., non-detectable activity.

FIG. 6 illustrates the effect of cosolvent and surfactant on the catalytic performance in converting 10 mM LUT by the coupled BsPPS/ErPPK biocatalytic system (6-mL) (c-d). The catalytic performance of the coupled system was evaluated by converting 10 mM LUT, 1 mM ATP, 20 mM MgCl₂, 20 mM polyP₆ with different cosolvent and surfactant, using 0.5 mg/mL crude BsPPS and crude ErPPK at 37° C. and 100 rpm in 200 mM Tris-HCl buffer (pH 7.5) in a 6-mL scale. Conversion rate (%) was calculated as the moles of LUT-P/the sum of the moles of LUT and LUT-Px 100. Data are mean±SD (n=3), with significance indicated by p<0.05 using t-test.

FIG. 7 illustrates the effect of BsPPS (a) and ErPPK (b) amounts on catalytic performance in the coupled BsPPS/ErPPK biocatalytic system. The 6-mL reaction mixture containing 10 mM LUT, 1 mM ATP, 20 mM MgCl₂, 20 mM polyP₆ with TWEEN 80 were incubated with crude BsPPS and crude ErPPK at 37° C. in 200 mM Tris-HCl buffer (pH 7.5) in a 6-mL scale. The conversion rate (%) was calculated as the moles of LUT-P/the sum of the moles of LUT and LUT-P×100. Data are mean±SD (n=3).

FIG. 8 illustrates conversion of 10 mM LUT by the coupled BsPPS/ErPPK biocatalytic system with pH control. The 80-mL reaction mixtures with 2.0 mg/mL crude BsPPS and 0.5 mg/mL crude ErPPK was conducted in 50 mM KPi buffer with pH maintained at pH 7.5±0.1 using 1 N NaOH. Conversion rate (%) was calculated as the moles of LUT-P/the sum of the moles of LUT and LUT-P×100.

FIGS. 9A and 9B illustrate conversion of selected polyphenol substrates by BsPPS and ErPPK. The reaction mixtures contained 2 mM polyphenol, 1 mM ATP, 0.5 mg/mL purified BsPPS and ErPPK, 20 mM MgCl₂, 20 mM polyP₆, 2% DMSO, and were incubated at 37° C. in 50 mM Tris-HCl buffer (pH 7.5, 100 mM NaCl). A conversion rate above 50% indicates the capability of ErPPK to regenerate ATP from AMP in the coupled enzymatic system.

FIG. 10 illustrates pH-controlled conversion of 20 mM LUT by the coupled BsPPS/ErPPK biocatalytic system. Time course of LUT conversion (a), HPLC chromatograms (b), and appearance of lyophilized LUT-P extract (c). The 80-mL reaction mixture containing 2.0 mg/mL crude BsPPS, 0.5 mg/mL crude ErPPK, 20 mM LUT, 1 mM ATP, 40 mM MgCl₂, 40 mM polyP₆ with TWEEN 80 was incubated at 37° C. and 400 rpm in 50 mM KPi buffer with pH controlled at pH 7.5±0.1 using 1 N NaOH. Conversion rate (%) was calculated as the moles of LUT-P/the sum of the moles of LUT and LUT-Px 100. L4′P, luteolin 4′-O-phosphate; L3′P, luteolin 3′-O-phosphate.

### DETAILED DESCRIPTION OF THE INVENTION

The detailed description and technical content of the present invention are now described as follows in conjunction with the drawings. Furthermore, the drawings in the present invention are not necessarily drawn according to the actual scale for the convenience of description. These drawings and their scales are not intended to limit the scope of the present invention.

Unless otherwise defined, all technical and scientific terms used herein have the same meaning as commonly understood by one of ordinary skill in the art to which this invention belongs. The following terms used throughout this application shall have the following meanings.

“Or” means “and/or” unless stated otherwise. “Comprising” means not excluding the presence or addition of one or more other components, steps, operations or elements to the described component, step, operation or element, respectively. The terms “comprising”, “including”, “containing”, and “having” as used herein are interchangeable and not limiting. As used herein and in the appended claims, the singular forms “a” and “the” include plural referents unless the context otherwise dictates. For example, the terms “a”, “the”, “one or more” and “at least one” are used interchangeably herein.

The terms “polypeptide,” “polypeptide fragment,” “peptide,” and “protein” are used interchangeably herein to refer to polymers of amino acid residues, and variants and synthetic analogs thereof. Accordingly, these terms apply to amino acid polymers in which one or more amino acid residues are synthetic non-naturally occurring amino acids, such as chemical analogs corresponding to naturally occurring amino acids. In certain aspects, polypeptides can include enzymatic polypeptides (or “enzymes”), which typically catalyze (i.e. increasing the rate of) various chemical reactions.

“Isolated”, as used herein, means that have been (1) separated (whether in nature and/or in an experimental context) from at least some of the components with which they were originally produced, and/or (2) engineered, produced, prepared and/or manufactured by humans. In some embodiments, a substance is “pure” if it is substantially free of other components. In some embodiments, as understood by those skilled in the art, a substance may still be considered “isolated” or even “pure” after being combined with certain other components such as one or more carriers or excipients (e.g. buffers, solvents, water, etc.); in such embodiments, the isolation or purity percentage of the material is calculated without the inclusion of such carriers or excipients. By way of example only, in some embodiments, a biopolymer such as a polypeptide or polynucleotide that occurs in nature is considered “isolated” when a) its source or origin is not the same as that found in nature in its native state associated with some or all of the components accompanied; b) it is substantially free of other polypeptides or nucleic acids from the same species as the species in which it is produced in nature; or c) expressed by cells of a species not in which it is produced in nature or other expression system or otherwise associated with components from said cell or other expression system. Thus, for example, in some embodiments, a polypeptide that is chemically synthesized or synthesized in a cell system different from the cell in which it is produced in nature is considered an “isolated” polypeptide. Alternatively or additionally, in some embodiments, a polypeptide that has been subjected to one or more purification techniques may be considered an “isolated” polypeptide to the extent that it has been separated from other components that: a) are associated in nature and/or b) are associated with which it was originally produced.

“Conserved”, as used herein, refers to the situation in biology that is similar or identical within a nucleic acid sequence, protein sequence, protein structure or polysaccharide sequence; it may occur between species, or between different molecules arising from the same organism. From an evolutionary point of view, it means a state in which a particular sequence continues to be preserved during the process of speciation.

The term “modified” does not necessarily mean that a nucleotide/amino acid analog or a non-natural nucleotide/amino acid is obtained by directly altering the natural nucleotides/amino acids, but that nucleotide/amino acid analogs or non-natural nucleotides/amino acids differ from natural nucleotides/amino acids. In some embodiments, the modification comprises chemical modification; when the embodiment involves genetic modification of microorganisms, typical strain development and/or molecular genetic techniques can be used to achieve the effect.

Polyphenolic phytochemicals are known as poor oral bioavailability due to their extremely low water solubility. In order to address the solubility and bioavailability issue, phosphate drugs is an alternative strategy. Phosphate drugs use phosphate synthase to hydrolysis into phosphorylated derivatives. However, the phosphorylation tools, phosphate synthase, are ATP-dependent, making a large requirement of ATP to processing the phosphorylation. In view of the demand for ATP by phosphate synthase, the present invention integrates the first enzyme with the second enzyme to form a coupled enzyme system. The first enzyme, polyphenol phosphorylation synthetase, is used to phosphorylate polyphenols, and the second enzyme is used to provide the ATP required by the first enzyme. Such cycle solves the problem that the phosphorylation process requires a large amount of ATP.

Therefore, the present invention relates to a coupled enzyme system, comprises: a first enzyme, which is a polyphenol phosphorylation synthetase; a second enzyme, which is an ATP regeneration enzyme; and a substrate being phosphorylated by the first enzyme.

In one aspect of the present invention, the first enzyme is an isolated or engineered polypeptide comprising a homologous protein sequence that is more than 70% identical to the polyphenol phosphorylation synthetase (SEQ ID NO: 1, MKKRGVSNMYSVLFRQAEESSQLAGAKGMNLIKLTKHGLPVPDGFIIQTNALAR FMEDNQLQETSENVESGIISGTFSDELKDELTSSFYKLRESYRSVAVRSSSASEDL EGASFAGQYETYLNIKTEEEFLAKVKECWASFFSGRVSSYKKKMNNQIAEPLMG IVVQGLIDSEMSGVIFSRNPVTHDDRELLISASYGLGEAVVSGSVTPDTFIVNKSSF EIQKEIGAKEIYMESAAEGIAEKETSEDMRSRFCLTDEQVIELAEITKKTEDLYGY PVDIEFGIADHQIYLLQARPITTIDQDKKAAEEKRSFMITDTDMNDFWLNMESNIE GPVSPLFSSFIVPALEYGLKKSMQKFPIGVVVDEVKLYRGHIYSKNQGGQQPPSE DCGKELFPILSEHMYDIINHTYLPFYRTLDQLAQTEHTAESALDAFQKLKAFYLT AYEEHFNIVFPQILLTNKLQAMYQDIQGESENAHFYEMLTGKMNKSLETDRCLW LFSMEVQENPNLLTIFENNKPEQLQEKLEQTDEGRHFLKNVHEFLQEYGWRSVK SHDLIEQIWVENPYFALANIQNYVRNGYHFDNEFQKTKEKREKLYNEFLENIEDP GLRTEFDRYYQWTLNSANIKDDHHFYIDAMLDAKARIFLLKIGELLAENGVIQDR EDLWFLYDDEVEQALLHPVSLQEKAEKRRQIFHEYELAQAPAYLGTPTKEQLKA AEEIVGAVIEDEKNTENHIFGIAASSGIATGPVKIIRDANEFSQFAPGDVLVCKMTT PLWTSLFQDAKAIITDTGGILSHAAIIAREYGIPAVLGTRTATERLRDGDIITVDGS SGKITVVSRS); wherein said polypeptide comprises a conserved domain which is based on the polyphenol phosphorylation synthetase (SEQ ID NO: 1) and sequentially comprises: an ATP-binding domain, comprising active catalytic sites of Lys27, Arg102, and Glu282; a substrate-binding domain, comprising a conserved motif of DDHHFYIDAMLDAKAR (SEQ ID NO: 2), and comprising active catalytic sites of Asp627, His629, and His630; and a phosphorylated histidine catalytic domain, comprising His795. In a preferred embodiment of the present invention, the first enzyme is polyphenol phosphorylation synthetase, more preferably, the first enzyme is Bacillus subtilis polyphenol phosphorylation synthetase (BsPPS).

The degree of identity between an isolated or engineered polypeptide and the amino acid sequence of SEQ ID NO: 1 is more than 70%; preferably at least 75%, more preferably at least 80%, even more preferably at least 85%; for example, the degree of identity is preferably more than 70%, more than 71%, more than 72%, more than 73%, more than 74%, more than 75%, More than 76%, more than 77%, more than 78%, more than 79%, more than 80%, more than 85%, more than 90%, more than 95% or more than 99%, etc. As used herein, “identity” of an amino acid sequence refers to the degree to which two sequences are mutually indistinguishable, and “similarity” refers to the same ratio and/or retention ratio between the two sequences. Those of ordinary skill in the art to which the present invention pertains should understand that the long-chain amino acids of polypeptides and proteins are only partially functional in their amino acid sequences, which are called functional motifs. Proteins have the same function when they have the same functional motif, in general, when the amino acid sequence of a polypeptide or protein is at least 40% identical, it has the same function (refer to How Proteins Work, Williamson, 2011). After alignment with homologous proteins or polypeptides, the amino acid sequence of the substrate-binding domain can be at least 70% identical to SEQ ID NO: 1, preferably at least 75%, more preferably at least 80%, even more preferably at least 85% identical amino acid sequences can have the same function.

A comparison between homologous proteins or polypeptides reveals that the amino acid sequences of the ATP-binding domain and of the phosphorylated histidine catalytic domain are relatively conserved in general and are not prone to much variation. The amino acid sequence of the ATP-binding domain is preferably SEQ ID NO: 3 (AGAKGMNLIKLTKHGLPVPDGFIIQTNALARFMEDNQLQETSENVESGIISGTFS DELKDELTSSFYKLRESYRSVAVRSSSASEDLEGASFAGQYETYLNIKTEEEFLAK VKECWASFFSGRVSSYKKKMNNQIAEPLMGIVVQGLIDSEMSGVIFSRNPVTHDD RELLISASYGLGEAVVSGSVTPDTFIVNKSSFEIQKEIGAKEIYMESAAEGIAEKET SEDMRSRFCLTDEQVIELAEITKKTEDLYGYPVDIEFGIADHQIYLLQARPITTIDQ DKKAAEEKR), and the amino acid sequence of the phosphorylated histidine catalytic domain is preferably SEQ ID NO: 4 (PGDVLVCKMTTPLWTSLFQDAKAIITDTGGILSHAAIIAREYGIPAVLGTRTATERL RDGDIITVDGSSG).

The polyphenolic phytochemical phosphate synthetase (PPS) of interest in the present invention has 839 amino acids (SEQ ID NO: 1), a molecular weight of 94.9 kDa, a pI value of 4.81, a Mowse score of 765, a protein sequence coverage of 31%, and a unique peptide sequence identified by protein mass spectrometry, with the gene of the target protein being a predicted protein gene (gene ID: 14103593) composed of 2520 bases. The nucleic acid sequence of the isolated, purified protein is known by way of gene cloning and DNA sequencing. More specifically, the nucleic acid sequences of those amino acid sequences capable of generating the substrate binding domain, the ATP binding domain, and the phosphorylated histidine catalytic domain are SEQ ID NO:5 (agcttcatgattaccgacactgatatgaatgatttctggcttaacatggagtctaatattgaaggtccggtgagtccgttattttcatc cttcatcgtgccggcattggaatatggcttgaagaagagcatgcaaaagtttccgattggtgtagttgttgatgaagtaaaactttat cgcggacatatttattccaaaaaccaaggtggacagcagcctccttctgaagactgcggcaaagagcttttcccgattttatcgga gcatatgtatgacatcatcaatcacacatacctccctttttaccggacactggaccagctcgcacaaactgagcataccgcagaaa gcgcactggatgcttttcaaaaactaaaggccttttatctcacggcttatgaagagcacttcaatatcgttttcccgcaaatccttttaa caaacaaactgcaagcgatgtatcaggacattcaaggagagtccgaaaacgctcatttttatgagatgctgacaggaaaaatgaa caaatcactggaaacggaccgttgcttatggctattttctatggaagttcaggagaacccgaaccttctgaccatttttgaaaacaa caagcctgaacagctccaggagaaattagaacaaacagatgaggggagacacttcctgaagaacgtccatgaattcttgcaag aatacggatggagatctgttaaaagtcatgatctgattgaacaaatctgggtggaaaatccgtatttcgctctggctaatattcaaaa ttatgtccgtaatggctatcattttgacaatgaatttcagaaaacgaaagaaaaacgagagaaattatacaatgaattcttggaaaac atagaagatcccggtttgcgcaccgaatttgaccgctattatcaatggacactgaactctgcaaatataaaagatgatcaccactttt atattgacgccatgctggatgccaaggcgagaatctttctgctgaagataggtgaattgctggcggaaaacggtgtcattcaagat cgtgaggacctttggtttttatatgacgacgaagtggaacaagcgcttcttcaccctgtatccctgcaagaaaaggctgaaaaacg cagacagatttttcatgagtatgagctggcccaagcccctgcctacctcggcaccccgacaaaagaacagctcaaagcagctga agaaattgtcggcgctgtgatagaggatgaaaaaaacacagagaatcatatttttggcattgcggcatcaagcggcattgcgaca ggtccggtgaaaatcattcgggacgccaatgaattttctcaattcgcg), SEQ ID NO: 6 (atgaagaaaagaggggtttcaaatatgtattctgttttatttcgccaggcagaagagtccagccagctggctggagcaaaagga atgaatttgattaaattgaccaaacacggtcttcctgttccggacgggtttattattcaaacgaatgcgctcgcacgttttatggagga caaccagcttcaagagactagtgaaaacgtcgaaagcgggatcatttctggaacattttcggatgagctgaaagatgagctgact agttccttttataagcttagagaatcatatcgatccgtagccgtgcgttcttcgtctgcttcggaagatttagaaggcgcctcattcgc gggtcaatatgaaacctacttaaatatcaaaacagaggaagagtttctggctaaagtgaaagaatgctgggcctcatttttttctgg gcgggtcagcagctataagaaaaaaatgaacaatcaaatcgcagagccgttaatgggaatagtcgttcaggggctgatcgattc agaaatgtcaggtgttatcttcagccgcaaccctgttacccatgatgatagagagcttttaatcagcgccagctacgggttgggtg aagctgttgtttcaggaagtgttaccccagacacgttcattgttaataaatcttcgtttgagattcagaaagaaataggtgcaaagga aatctacatggagtctgcggcagaaggaattgctgaaaaagaaacgagtgaagacatgcgcagccgtttttgccttacagatga acaagtgattgaattggctgaaatcacaaaaaaaaccgaagacctgtacggatatcctgtcgatatagaatttggaattgctgatca tcaaatataccttctgcaagctcgcccgattacaaccattgatcaggacaaaaaggcggcagaagaaaaacgc), and SEQ ID NO: 7 (cctggggacgtactcgtttgcaagatgaccacaccgctatggaccagcctgtttcaagacgccaaagcgataattacagacac aggcggcattttgtctcacgctgcgattattgcccgtgaatacggcattccagccgttctcggcacacgcacggcaaccgaaaga ctgcgagacggtgacatcatcactgttgacggtagcagcggcaaaatcacagttgtcagccggtcctga) respectively.

The phosphorylation stated herein refers to the addition of a phosphate group to a protein or another type of molecule. This reaction plays an important role in energy metabolism and signal transduction in a living body and is a critical to biochemistry. Currently known phosphorylation entails kinase (which is a phophotransferase) or phosphorylase, both falling within the EC 2.7 category, and the reaction requires ATP as the source of energy and Mg²⁺ ions as a cofactor. Generally, the aforesaid enzymes hydrolyze ATP and transfer 7-phosphate to the substrate. Protein kinase is the most common large-molecule phosphorylation enzyme, is responsible for modifying, through phosphorylation, a wide range of proteins with different functions, and is an essential means for regulating signal transduction in a living body. More and more physiological phenomena, such as whether an enzyme is activated or not, have been found to be related to the phosphorylation or dephosphorylation of protein. Certain amino acid sites on a protein molecule, such as the —OH functional group of serine, threonine, or tyrosine, or the imidazole ring of histidine, can be modified by protein kinase through phosphorylation such that the molecule is activated by the addition of phosphoric acid, and this phosphoric acid can be subsequently removed with protein phosphatase to render the molecule deactivated. There are also many examples in which similar reactions produce the opposite effects. As to small-molecule phosphorylation enzymes such as acetokinase, glycerokinase, arginine kinase, shikimate kinase, mevalonate kinase, and nucleoside kinase, they are responsible for such crucial catalytic reactions in the metabolic pathways in a living body as glycolysis, the biosynthesis of amino acids, the biosynthesis of cholesterol, and the biosynthesis of nucleotides. Phosphorylation enzymes can also be divided by the source of the phosphate group into the following two types. The first type uses a phosphoric acid monoester as the phosphate donor and is generally capable of hydrolyzing ATP and transferring γ-phosphate to the substrate. The second type uses a phosphonate diester or pyrophosphate as the phosphate donor instead.

Regarding the catalytic sites stated herein, the inventor of the present invention has found a number of important catalytic sites through an extensive research, including Lys27, which is related to ATP binding, and His795, which is responsible for carrying and transferring the phosphate group. In some embodiments, the ATP-binding domain includes such catalytically active sites as Lys27, Arg102, and Glu282, all of which are related to ATP binding. In some other embodiments, the phosphorylated histidine catalytic domain includes His795, which is a catalytic site that carries and transfers the phosphate group. As for the important catalytic sites in the substrate-binding domain, the inventor has found that the catalytically active sites Asp627, His629, and His630 in the substrate-binding domain, as well as His795 (which is responsible for carrying and transferring the phosphate group) in the phosphorylated histidine catalytic domain, of the polypeptide in question are important active sites in the catalysis of phosphorylation. Under certain circumstances, the substrate-binding domain includes a conserved sequence whose amino acid sequence is DDHHFYIDAMLDAKAR (SEQ ID NO: 2).

The first enzyme of the system of the present invention can catalyze a substrate to their phosphate derivatives. Said substrate may be a polyphenolic phytochemical. In an embodiment of the present invention, the substrate is selected from the group consisting of the following formulas:

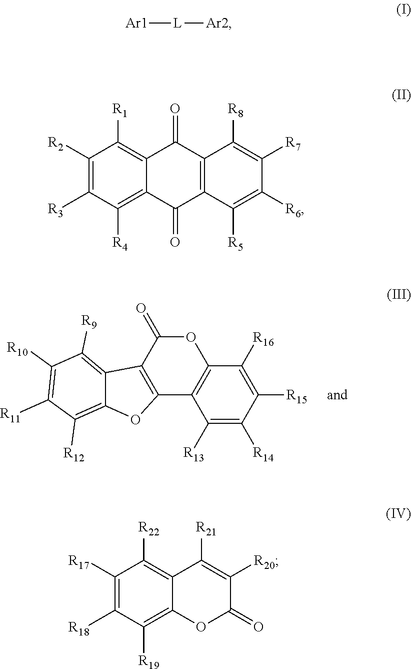

wherein Ar1 is an aryl group of the following formula:

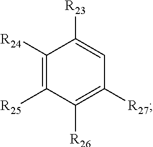

Ar2 is an aryl group of the following formula:

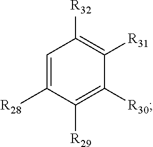

L is a linking group comprising 3 to 7 backbone carbon atoms forming a chain linking Ar1 and Ar2 as the case may be, wherein L comprises at least one of a double bond, a carbonyl group and a hydroxyl group;
R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;
R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁)phenyl group or (C₇-C₁₂)benzyl group, wherein q is an integral of 1 to 3, and at least one of R₉ to R₁₆ is a hydroxyl group;
R₁₇ to R₂₂ are respectively H, methoxy group or hydroxyl group, and at least one of R₁₇ to R₂₂ is a hydroxyl group, or R₂₀ and R₂₁, R₁₇ and R₁₈, R₁₇ and R₂₂, R₁₈ and Rig or their combination are fused to form a (C₃-C₆)cycloalkyl group with hydroxyl group or a (C₆-C₁₀)aryl group with hydroxyl group;
R₂₃ to R₂₇ are respectively H, methoxy group or hydroxyl group, and at least one of R₂₃ to R₂₇ is a hydroxyl group;
R₂₈ to R₃₂ are respectively H, methoxy group or hydroxyl group, and at least one of R₂₈ to R₃₂ is a hydroxyl group;
R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₅ is (C₁-C₅)alkyl group, (C₁-C₅)haloalkoxy group, (C₆-C₁₁)phenyl group or (C₇-C₁₂)benzyl group;
R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₇ to R₄₀ are respectively (C₁-C₅)alkyl group or (C₁-C₅)haloalkoxy group;
R₄₁ and R₄₂ are respectively H, (C₁-C₅)alkyl group or (C₁-C₅)haloalkoxy group, one of which is H and the other is not H;
R₄₃ and R₄₄ are respectively H, (C₁-C₅)alkyl group or (C₁-C₅)haloalkoxy group.

In an embodiment of the present invention, the second enzyme is ATP regeneration enzyme, preferably a polypeptide having a sequence that is more than 65% (for example, more than 70%, more than 71%, more than 72%, more than 73%, more than 74%, more than 75%, More than 76%, more than 77%, more than 78%, more than 79%, more than 80%, more than 85%, more than 90%, more than 95% or more than 99%) identical to polyphosphate Kinase 2 Class III (PPK2-III), and more preferably, the second enzyme is ErPPK. ATP regeneration enzyme is capable of transferring the phosphate group of polyphosphate (polyP), a. cost-effective phosphate donor, to nucleotides such as AMP or ADP, thus generating ATP. Given that polyphenol phosphorylation synthetase uses one mole of polyphenolic phytochemicals, ATP, and water to yield one mole of monophosphate of polyphenolic phytochemicals, AMP, and Pi, ATP regeneration enzyme is suited for coupling with polyphenol phosphorylation synthetase, enabling in vitro ATP recycling from AMP, and constructing a bienzymatic system. In the systems, mesophilic or thermophilic PPK2-III have been used with different ATP-dependent enzymes, including AaPPK (SEQ ID NO: 8, MPMVAAVEFAKSPAEVLRVGSGFSLAGVDPESTPGYTGVKADGKALLAAQDAR LAELQEKLFAEGKFGNPKRLLLILQAMDTAGKGGIVSHVVGAMDPQGVQLTAF KAPTDEEKSHDFLWRIEKQVPAAGMVGVFDRSQYEDVLIHRVHGWADAAELER RYAAINDFESRLTEQGTTIVKVMLNISKDEQKKRLIARLDDPSKHWKYSRGDLAE RAYWDDYMDAYSVAFEKTSTEIAPWHVVPANKKWYARIAVQQLLLDALGGLQ LDWPKADFDVAAERALVVES) from A. aurescens, DpPPK (SEQ ID NO: 9, MNSKASQQLQVPPGQKVRLADYSTDTFEGSAALDKEQVKQATEQLQQRLSELQ EKLYAEGKQSLLIILQARDGGGKDSTVSRVMGAFNPNGVHVANFKAPTDLELQH DFLWRIHQQVPSHGMIAVFNRTSHYEDVLVTRVHGLIDDARAQQNLEHIVNFEKL LSDAGTRIVKFYLHLSPEEQKARMEDRLNDPAKHWKFNPSDLKDRALWHEYTA AYEDALATSRSYAPWYIIPADRKWLRDYLISEILVQTLEEMNPQFPTAHFEAAYY LIEDIPSR) from D. proteolyticus, ChPPK (SEQ ID NO: 10, MATDFSKLSKYVETLRVKPKQSIDLKKDFDTDYDHKMLTKEEGEELLNLGISKLS EIQEKLYASGTKSVLIVFQAMDAAGKDGTVKHIMTGLNPQGVKVTSFKVPSKIEL SHDYLWRHYVALPATGEIGIFNRTSHYENVLVTRVHPEYLLSEQTSGVTAIEQVNQ KFWDKRFQQINNFEQHISENGTIVLKFFLHVSKKEQKKRFIERIELDTKNWKFSTG DLKERAHWKDYRNAYEDMLANTSTKQAPWFVIPADDKWFTRLLIAEIICTELEK LNLTFPTVSLEQKAELEKAKAELVAEKSSD) from C. hutchisonii, or ErPPK (SEQ ID NO: 11, MINIYKIDKLNNFNLNNHKTDDYSLCKDKDTALELTQKNIQKIYDYQQKLYAEK KEGLIIAFQAMDAAGKDGTIREVLKALAPQGVHEKPFKSPSSTELAHDYLWRVH NAVPEKGEITIFNRSHYEDVLIGKVKELYKFQNKADRIDENTVVDNRYEDIRNFE KYLYNNSVRIIKIFLNVSKKEQAERFLSRIEEPEKNWKFSDSDFEERVYWDKYQQ AFEDAINATSTKDCPWYVVPADRKWYMRYVVSEIVVKTLEEMNPKYPTVTKET LERFEGYRTKLLEEYNYDLDTIRPIEK) from an unclassified Erysipelotrichaceae bacterium.

In the coupled enzyme system, the first enzyme catalyzes the substrate first and yields phosphate derivative, AMP and Pi; the second enzyme uses the AMP produced from the first enzyme to ATP so that regenerating ATP and constructing the ATP recycle. The inventor conducts a series of experiments such as temperature, pH dependence and stability to investigate the impact of the first enzyme on the second enzyme. The inventor found that the reaction temperature of the first enzyme and the second enzyme is 35 to 40° C., such as but not limited to 35° C., 35.1° C., 35.2° C., 35.3° C., 35.4° C., 35.5° C., 35.6° C., 35.7° C., 35.8° C., 35.9° C., 36° C., 36.1° C., 36.2° C., 36.3° C., 36.4° C., 36.5° C., 36.6° C., 36.7° C., 36.8° C., 36.9° C., 37° C., 37.1° C., 37.2° C., 37.3° C., 37.4° C., 37.5° C., 37.6° C., 37.7° C., 37.8° C., 37.9° C., 38° C., 38.1° C., 38.2° C., 38.3° C., 38.4° C., 38.5° C., 38.6° C., 38.7° C., 38.8° C., 38.9° C., 39° C., 39.1° C., 39.2° C., 39.3° C., 39.4° C., 39.5° C., 39.6° C., 39.7° C., 39.8° C., 39.9° C., or 40° C. Also, the reaction pH value of the first enzyme and the second enzyme is 7.0 to 8.0, such as but not limited to 7.0 to 7.5, 7.0 to 7.8, 7.2 to 7.9, 7.2 to 7.6, 7.3 to 7.8, 7.3 to 7.7, 7.4 to 7.6, 7.4 to 7.8, 7.5 to 7.6, 7.5 to 7.7, 7.5 to 7.8, 7.6 to 7.7, 7.6 to 7.8, or 7.7 to 7.8.

In phosphotransferases, divalent metal ions such as Mg²⁺ or Mn²⁺ are essential for coordinating phosphate-containing molecules within the active site of enzyme. The stoichiometry of the divalent metal ion relative to the phosphate donor plays an important role in modulating enzyme phosphorylation activity. In the system of the present invention, which includes both the first enzyme and the second enzyme, phosphotransferases that depend on Mg²⁺ or Mn²⁺, the Mg²⁺ or Mn²⁺ to polyP ratio may be crucial for optimal enzyme activity. In an embodiment of the present invention, the coupled biocatalytic system further comprises Mg²⁺ as divalent cation preference and polyphosphate (polyP) as phosphate donor. In a preferred embodiment of the present invention, Mg²⁺ to polyP ratio is 1:1.

In enzyme technology, solubilization of substrates using cosolvents or surfactants is crucial for the enzymatic conversion of insoluble substrates. However, these agents can affect enzyme stability by disrupting molecular interactions essential for protein folding. This necessitates identifying agents that solubilize effectively while minimally impacting enzyme stability, to enhance production yields. In an embodiment of the present invention, our system further comprises a cosolvent and a surfactant. The cosolvent is, for example but not limited to, DMSO, propylene glycol, polyethylene glycol, and glycerine. The surfactant is, for example but not limited to, sodium dodecyl sulphate (SDS), sodium lauryl sulphate (SLS), polyoxyethylene sorbitan fatty acid esters (Tweens), polyoxyethylene stearates, sorbitan fatty acid esters (Spans), polyethylene glycol 15 hydroxystearate, and Triton. In a preferred embodiment of the present invention, the surfactant is TWEEN 20 or TWEEN 80.

Another objective of the present invention is to provide a method for synthesizing a polyphenolic phytochemicals phosphate derivative, comprises steps of: (a) providing a polyphenol phosphorylation synthetase, an ATP regeneration enzyme, and a polyphenolic phytochemical as a substrate; (b) catalyzing the polyphenolic phytochemical to monophosphates and yield AMP and Pi by the polyphenol phosphorylation synthetase; (c) recycling ATP by the ATP regeneration enzyme from AMP which was yielded by the polyphenol phosphorylation synthetase; (d) continuously catalyzing the substrate by the polyphenol phosphorylation synthetase to obtain the polyphenolic phytochemicals phosphate derivative.

The method of the present invention for synthesizing a polyphenolic phytochemical phosphate derivative can transform a polyphenolic phytochemical into its phosphate derivatives. Those polyphenolic phytochemical phosphate derivatives have a higher absorption rate and higher bioavailability than non-phosphorylated polyphenolic phytochemicals and, thanks to their advantageous bioactivity, can be used to make food, pharmaceuticals, industrial materials, and so on. Some examples of the aforesaid food are nutritional supplements, health food, functional food, baby food, and food for the elderly. Such food may be a solid, a fluid, a liquid, or a mixture of the above, preferably a liquid. When a pharmaceutical (e.g., a prodrug) is made, there is no special limitation on its dosage form. For example, the dosage form may be a solution, a paste, a gel, a solid, powder, or any other forms. If necessary, the pharmaceutical may include another pharmaceutically active ingredient (e.g., an anti-inflammation ingredient) or an auxiliary ingredient (e.g., a lubricating ingredient or a vehicle ingredient).

### SPECIFIC EXAMPLES

The example is divided into six parts. First, a suitable second enzyme, PPK2-III enzyme, is screened out, and assessed whether ErPPK is compatible with the first enzyme, polyphenol phosphorylation synthetase (using BsPPS as example). Next, after the optimal ratio of Mg²⁺ and polyphosphate was found, co-solvents and surfactants were added to test their impact on the conversion of the system of the present invention. The example also tested the effects of different concentrations of BsPPS and ErPPK and pH control on the system. Finally, the catalytic performance of the two enzyme system (BsPPS/ErPPK) was assessed for synthesis of polyphenol phosphates.

### 1. Screening PPK2-III Enzymes and ErPPK Characterization

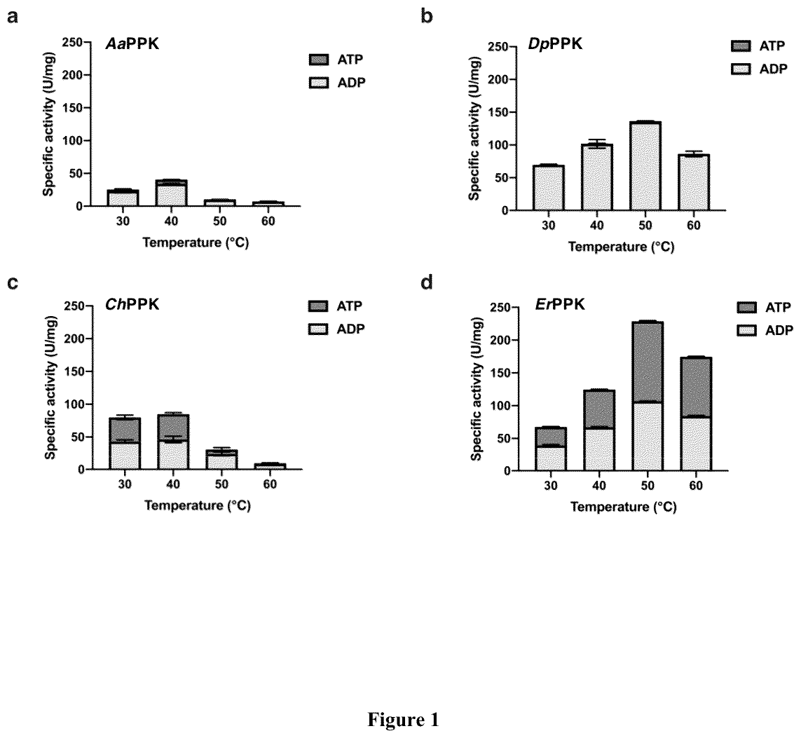

AaPPK from A. aurescens, DpPPK from D. proteolyticus, ChPPK from C. hutchisonii, and ErPPK from an unclassified Erysipelotrichaceae bacterium were selected as PPK2-III candidates for ATP regeneration from AMP (Table 1). Their selection was based on operating conditions at temperatures of 30 and 37° C., and pH values of 7.5 and 8.0, which may align with the optimal conditions of BsPPS (40° C., pH 7.8, Mg²⁺/Mn²⁺). Temperature dependence of these candidates under pH 7.5 was investigated using AMP as the substrate to identify the most effective enzyme for ATP regeneration alongside BsPPS. As seen in FIG. 1, all candidates showed polyP-dependent AMP phosphorylation activity, with optimal performance at 40° C. or 50° C. ErPPK exhibited the highest ATP synthetic activity from AMP at 40° C., followed by ChPPK, while AaPPK, and DpPPK had low ATP formation rates. DpPPK showed significant AMP phosphorylation activity, but predominantly yielded ADP with limited ATP formation. Notably, although ErPPK has been previously used at 30° C. and pH 7.5, our findings indicate that its optimal temperature is 50° C. and maintained high activity at 40° C., making it a suitable partner for BsPPS.

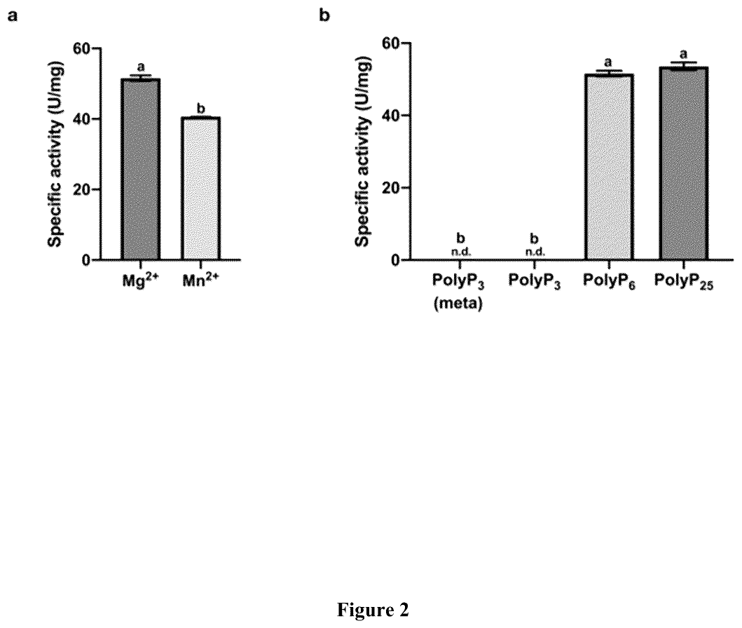

As seen in FIG. 2, ErPPK could use both Mg²⁺ and Mn²⁺ as cofactor with a preference for Mg²⁺, similar to BsPPS, and efficiently utilized sodium hexametaphosphate (polyP₆), a cost-effective and readily available phosphate donor. Therefore, ErPPK was selected for further biochemical characterization to evaluate its compatibility with BsPPS.

TABLE 1
PPK2-III candidates used in the present invention.

| PPK2- | | | Length | Mass |
|---|---|---|---|---|
| III | Source | Classification | (aa) | (kDa) |
| AaPPK | Anthrobacter | G+, Mesophile | 286 | 31.658 |
| | aurescens | | | |
| ChPPK | Cytophaga | G−, Mesophile | 305 | 35.252 |
| | hutchinsonii | | | |
| DpPPK | Deinococcus | G+, Mesophile | 277 | 31.845 |
| | proteolyticus | | | |
| ErPPK | Erysipelotrichaceae | — | 298 | 35.543 |
| | bacterium (biogas | | | |
| | fermenter metagenome) | | | |

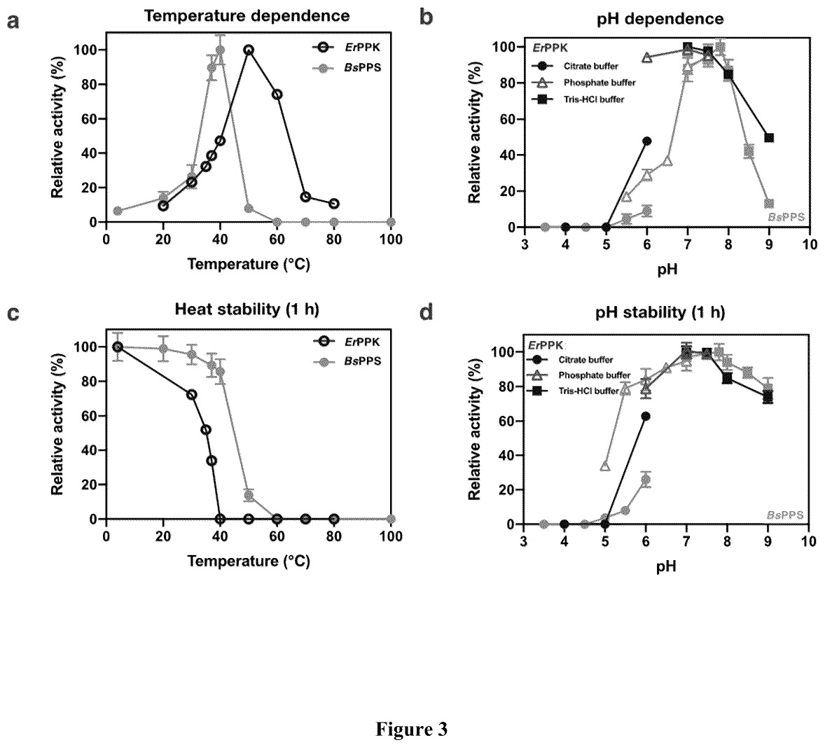

In investigating reaction conditions comprising BsPPS and ErPPK, we characterized the biochemical properties of ErPPK in comparison with BsPPS. ErPPK displayed the highest activity at 50° C. and pH 7.0, with moderate activity at 40° C. and pH 7.8 (FIGS. 3a and 3b). However, ErPPK exhibited low stability at 40° C., losing activity after 1 h incubation. ErPPK maintained moderate activity and stability at 35° C. and 37° C., whereas BsPPS had a narrow temperature range with a sharp decrease in activity at 35° C. (FIG. 3c). Therefore, 37° C. was identified as the best compromise between the activity and stability of both enzymes. Based on the biochemical properties of BsPPS and ErPPK, we constructed the bienzymatic system using Mg²⁺ as the required cofactor and polyP₆ as the phosphate donor for ErPPK, operating at 37° C. and pH 7.5.

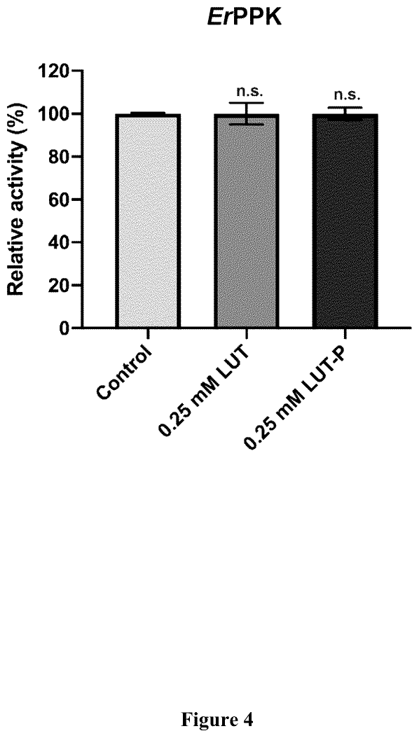

Considering the known inhibitory effect of aromatic phosphonates on PPK2 enzymes, we investigated the impact of polyphenols and polyphenol phosphates on ErPPK activity to evaluate their integration into a one-pot bi-enzymatic system. LUT was selected as the polyphenol target, based on its incomplete conversion in microbial systems due to its toxic nature. At a concentration of 0.25 mM, both LUT and its monophosphates showed no significant inhibitory effect on ErPPK activity (FIG. 4). The absence of a significant inhibitory effect of both LUT and its phosphate derivatives on ErPPK activity suggests the suitability of ErPPK for inclusion in the coupled enzyme system.

### 2. Effect of Mg²⁺ and Polyphosphate

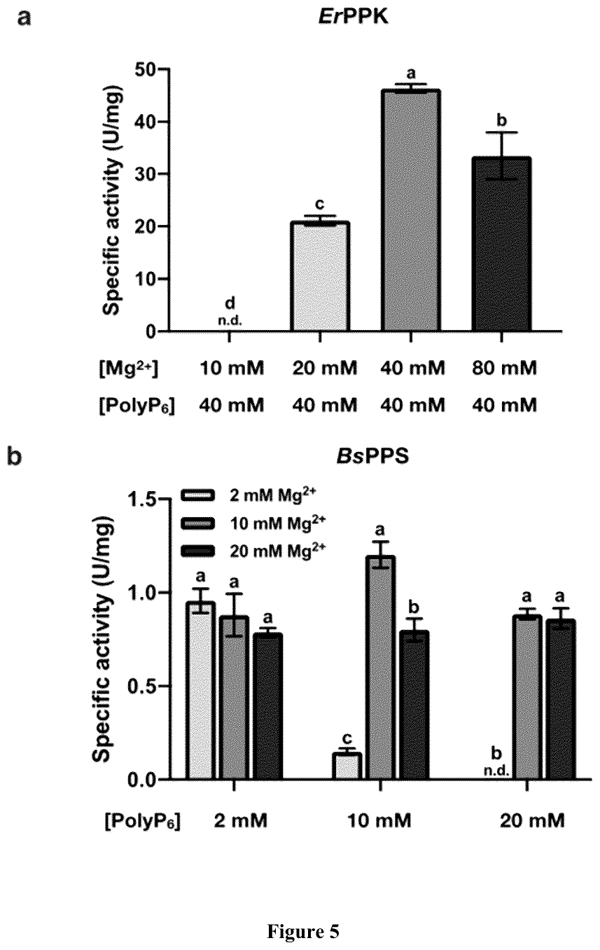

In our system, which includes both ErPPK and BsPPS, phosphotransferases that depend on Mg²⁺ or Mn²⁺, the Mg²⁺ to polyP ratio may be crucial for optimal enzyme activity. Our investigation into the impact of this ratio revealed that a 1:1 Mg²⁺/polyp ratio resulted in the highest activity for both enzymes (FIG. 5). Excess polyP, a substrate for ErPPK, decreased the activities of both ErPPK and BsPPS, but these activities were restored with an equimolar supplementation of Mg²⁺.

These findings are consistent with previous studies indicating the importance of the Mg²⁺ to polyP ratio for PPK2-III activity. The observed decrease in BsPPS activity within the one-pot system is likely due to imbalances in the Mg²⁺/polyP ratio, leading to a reduction in available Mg²⁺ ions crucial for forming the active enzyme-Mg²⁺-ATP complex. This suggests the importance of maintaining a balanced Mg²⁺/polyP ratio. Consequently, we controlled a 1:1 ratio of Mg²⁺ to polyP in the coupled system.

### 3. Enhancing Substrate Accessibility Using TWEEN Surfactants

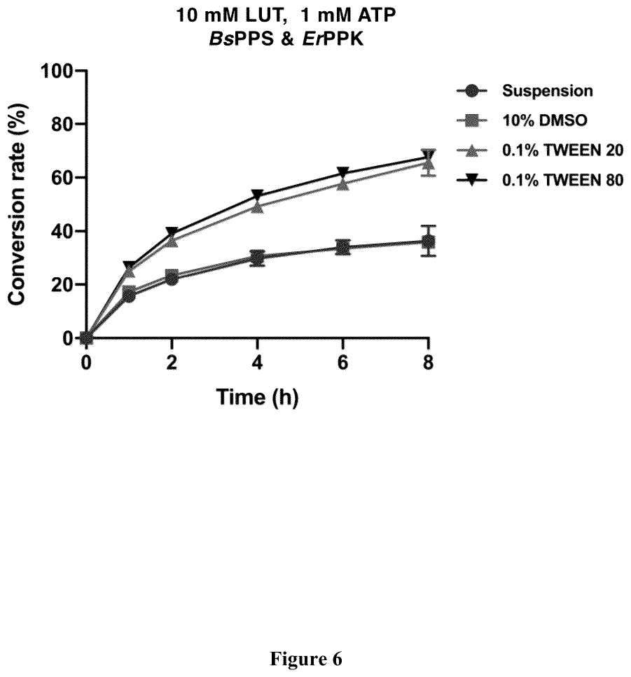

Due to the effect of cosolvents and surfactants on increasing substrate accessibility to the active site of enzyme, we incorporated solubilizing agents including DMSO, TWEEN 20, and TWEEN 80 into the BsPPS/ErPPK biocatalytic system, and assessed their effect on converting 10 mM LUT, our selected polyphenol target, with 1 mM ATP. The control group, without solubilizing agents, exhibited a low conversion rate (FIG. 6). Though adding 10% DMSO improved substrate solubility, it may decrease enzyme stability, thereby limiting the conversion. In contrast, both TWEEN surfactants achieved significantly higher conversion rates, with TWEEN 80 slightly outperforming TWEEN 20. This emphasizes the importance of solubilizing agents in enzymatic activity for efficient conversion of poorly water-soluble polyphenols.

### 4. Optimizing Enzyme Ratios

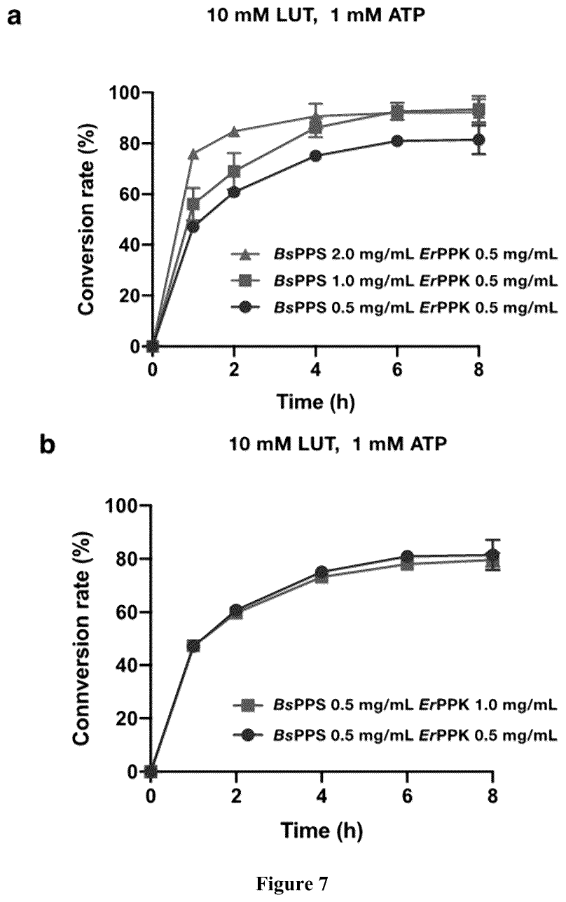

In the coupled biocatalytic system comprising two crude enzymes, BsPPS for polyphenol phosphorylation and ErPPK for ATP regeneration, we aimed to optimize enzyme ratios. In evaluating the effects of varying enzyme amounts, we discovered that increasing the amount of BsPPS led to an enhanced conversion rate, while additional ErPPK had no significant impact (FIGS. 7a and 7b). This indicated that BsPPS is the rate-limiting enzyme in the system. By optimizing the concentration of the rate-limiting enzyme, BsPPS, we achieved a conversion rate of 92.2% using 2.0 mg/mL BsPPS and 0.5 mg/mL ErPPK after 8 h. This conversion rate was achieved in the presence of TWEEN 80, using 10 mM LUT and 1 mM ATP as initial substrates.

### 5. Role of pH Control

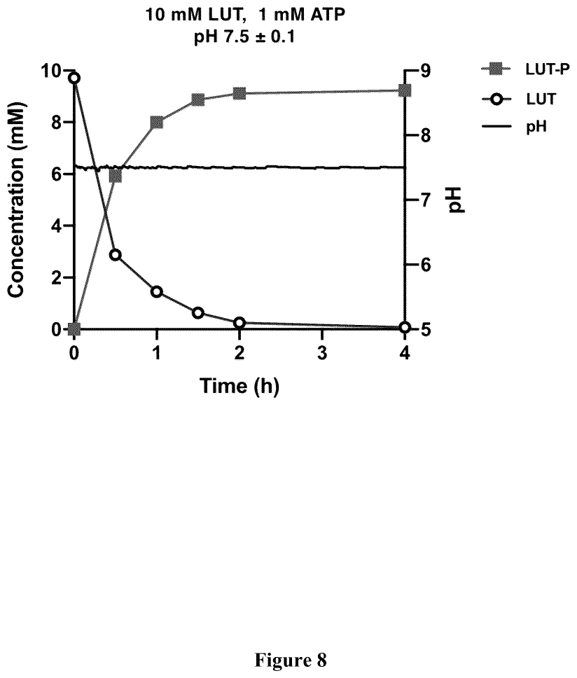

The aforementioned use of surfactants to enhance substrate solubility, combined with an increase in the rate-limiting enzyme BsPPS, led to improved conversion rates, though complete conversion was not achieved. A notable observation was the gradual decline in pH during the conversion process, which could potentially affect catalytic performance. To investigate the effect of pH changes on LUT conversion, 10 mM LUT was converted in 50 mM potassium phosphate (KPi) buffers, with pH controlled at 7.5. By implementing a pH stat strategy, rapid conversion of LUT was achieved, with complete conversion within 2 h (FIG. 8). This points to the necessity for pH control in biocatalytic systems involving BsPPS and ErPPK to maintain optimal enzymatic activity and conversion efficiency.

### 6. Synthesis of Polyphenol Phosphates

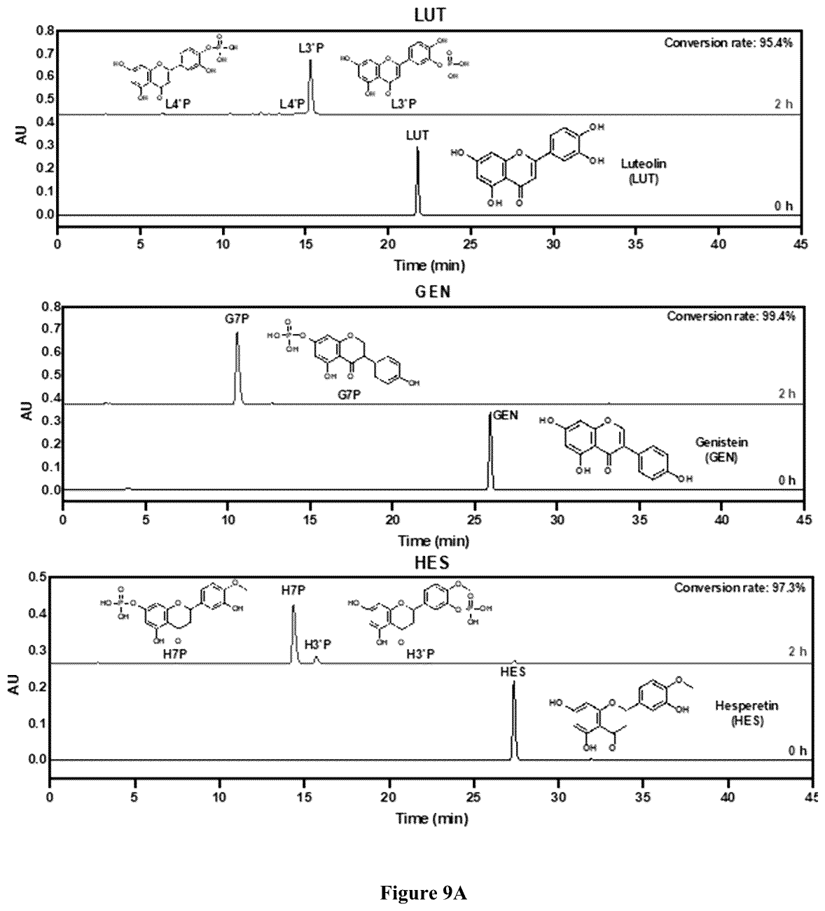

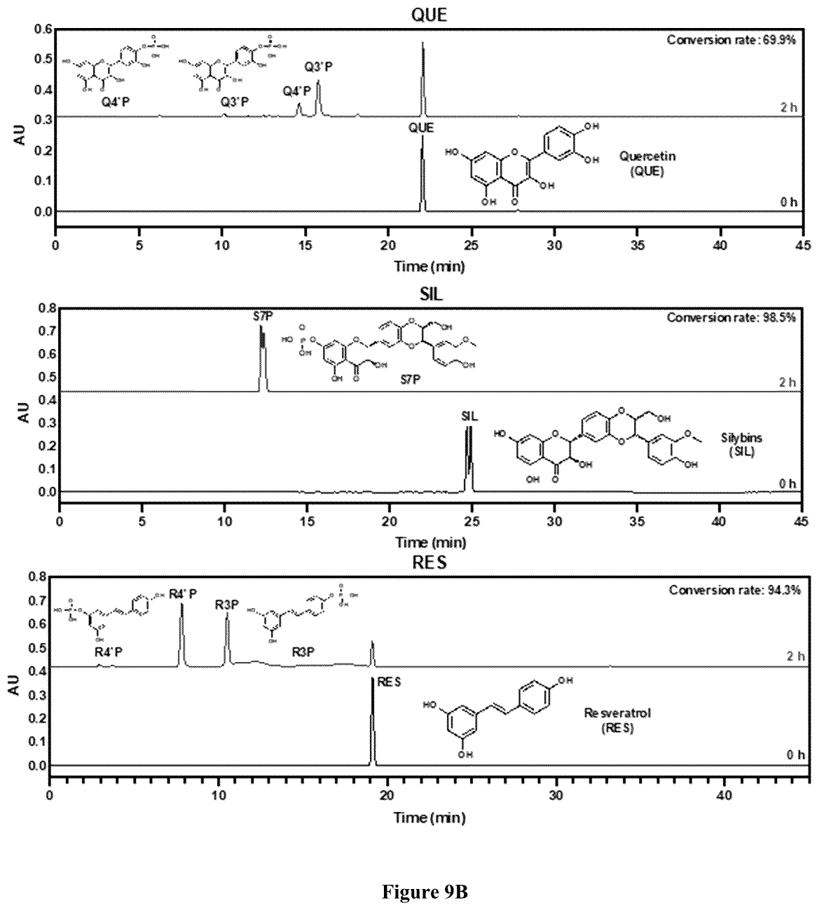

To test the versatility of this system, we used ErPPK and BsPPS to catalyze the conversion of 2 mM of various polyphenol substrates, including LUT, genistein, hesperetin, quercetin, silybins, and resveratrol with 1 mM ATP. Considering the stoichiometry, a conversion rate exceeding 50% indicates the ATP regeneration capability of ErPPK in the system. As seen in FIG. 9, in all tested cases, all the conversion rates surpassed 50%, with most exceeding 94% after a 2-h incubation period. This suggests that ErPPK effectively supports ATP generation in the coupled system across a range of polyphenol substrates.

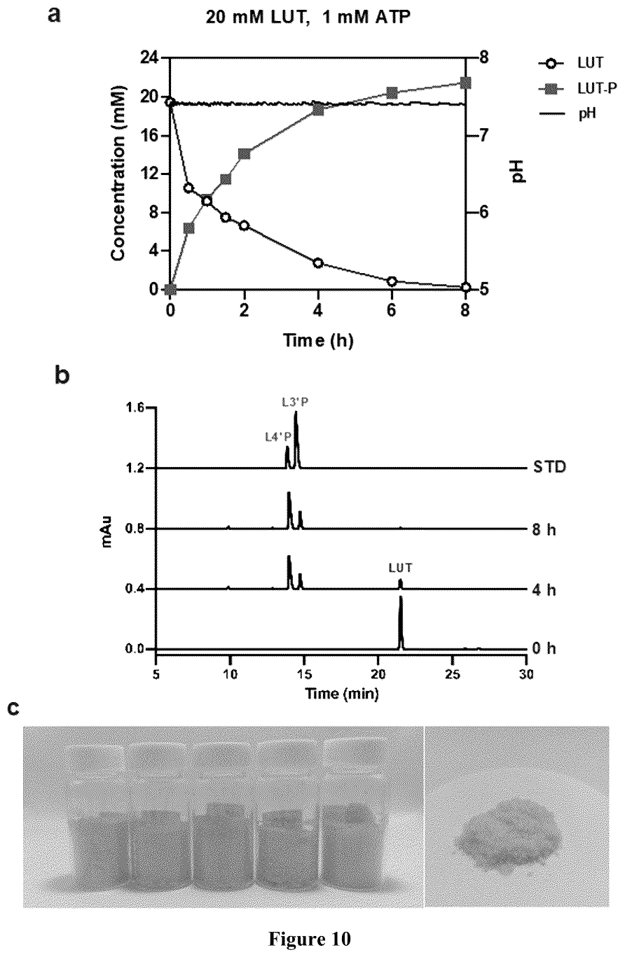

To investigate conversion of LUT at a higher concentration, we employed the pH-stat approach, maintaining the pH at 7.5 using NaOH in 50 mM KPi buffer. This experiment involved 20 mM LUT and 1 mM ATP. Effective control of pH enabled complete conversion with a high conversion rate of 99.0% within 8 h, yielding up to 21.5 mM LUT-P (FIGS. 10a and 10b). Notably, the highest productivity was achieved at 4 h, reaching 4.7 mM/h and yielding 18.6 mM LUTP, corresponding to an 87.2% conversion rate (FIG. 10a). Remarkably, this was achieved with an initial concentration of 21.7 mM LUT and only 1 mM ATP, reducing ATP use by up to 95%. This performance significantly surpasses previous microbial conversion methods, which only achieved a maximum conversion rate of 44.3% after 48 h and a much lower productivity of 0.2 mM/h. Following the reaction, extraction with ethyl acetate and lyophilization of the reaction mixture yielded a yellow powder of LUT-P extract with 72.4% purity (FIG. 10c).

To sum up, our results achieved through an enzymatic approach that is free from biological constraints and adaptable to a wider range of polyphenol applications, demonstrate the superiority of enzymatic phosphorylation over microbial conversion in terms of efficiency and versatility. Consequently, it highlights the potential of enzymatic systems in the production of polyphenol monophosphates, presenting a promising pathway for the scalable synthesis of bioactive compounds in a water-soluble form.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A coupled enzyme system, comprises:
a first enzyme, which is a polyphenol phosphorylation synthetase;
a second enzyme, which is an ATP regeneration enzyme; and
a substrate, being phosphorylated by the first enzyme.

2. The coupled enzyme system of claim 1, wherein the first enzyme is an isolated or engineered polypeptide comprising a homologous protein sequence that is more than 70% identical to SEQ ID NO: 1;
wherein said polypeptide sequentially comprises:
an ATP-binding domain, comprising active catalytic sites of Lys27, Arg102, and Glu282;
a substrate-binding domain, comprising a conserved motif of DDHHFYIDAMLDAKAR (SEQ ID NO: 2), and comprising active catalytic sites of Asp627, His629, and His630; and
a phosphorylated histidine catalytic domain, comprising His795.

3. The coupled enzyme system of claim 1, wherein the second enzyme is a polypeptide having a sequence that is more than 65% identical to polyphosphate kinase 2 Class III (PPK2-III), AaPPK from A. aurescens (SEQ ID NO: 8), DpPPK from D. proteolyticus (SEQ ID NO: 9), ChPPK from C. hutchisonii (SEQ ID NO: 10), or ErPPK from an unclassified Erysipelotrichaceae bacterium (SEQ ID NO: 11);
wherein said polypeptide sequentially comprises:
a conserved walker A (GXDXXGK) motif of AXDXXGK for coordinating the divalent cation and polyphosphate; a conserved walker B (DR) motif of NR or DR for coordinating the nucleotide phosphate groups; active catalytic sites of Asp66, Lys70, Arg192, and Asp206 in ErPPK (SEQ ID NO: 11) and equivalent residues.

4. The coupled enzyme system of claim 1, wherein the substrate is selected from the group consisting of the following formulas: 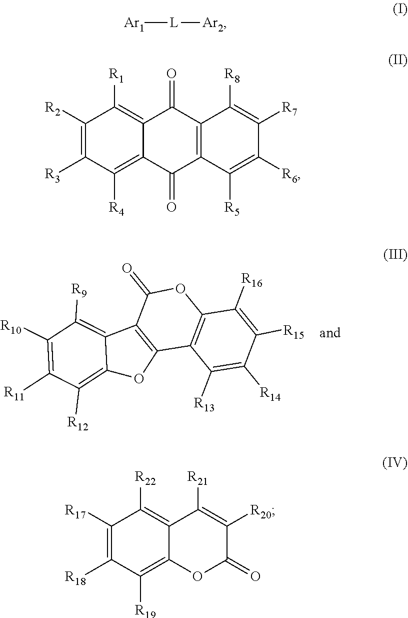
wherein Ar1 is an aryl group of the following formula: 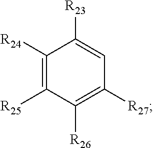
Ar2 is an aryl group of the following formula: 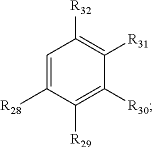
L is a linking group comprising 3 to 7 backbone carbon atoms forming a chain linking Ar1 and Ar2 as the case may be, wherein L comprises at least one of a double bond, a carbonyl group and a hydroxyl group;
R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;
R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₉ to R₁₆ is a hydroxyl group;
R₁₇ to R₂₂ are respectively H, methoxy group or hydroxyl group, and at least one of R₁₇ to R₂₂ is a hydroxyl group, or R₂₀ and R₂₁, R₁₇ and R₁₈, R₁₇ and R₂₂, R₁₈ and R₁₉ or their combination are fused to form a (C₃-C₆)cycloalkyl group with hydroxyl group or a (C₆-C₁₀)aryl group with hydroxyl group;
R₂₃ to R₂₇ are respectively H, methoxy group or hydroxyl group, and at least one of R₂₃ to R₂₇ is a hydroxyl group;
R₂₈ to R₃₂ are respectively H, methoxy group or hydroxyl group, and at least one of R₂₈ to R₃₂ is a hydroxyl group;
R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₅ is (C₁-C₅)alkyl group, (C₁-C₅)haloalkoxy group, (C₆-C₁₁)phenyl group or (C₇-C₁₂)benzyl group;
R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₇ to R₄₀ are respectively (C₁-C₅)alkyl group or (C₁-C₅)haloalkoxy group;
R₄₁ and R₄₂ are respectively H, (C₁-C₅)alkyl group or (C₁-C₅)haloalkoxy group, one of which is H and the other is not H;
R₄₃ and R₄₄ are respectively H, (C₁-C₅)alkyl group or (C₁-C₅)haloalkoxy group.

5. The coupled enzyme system of claim 1, wherein the reaction temperature of the system is 35 to 40° C.

6. The coupled enzyme system of claim 1, wherein the reaction pH value of the system is 7.0 to 8.0.

7. The coupled enzyme system of claim 1, further comprises a divalent metal ion and a polyphosphate (polyP).

8. The coupled enzyme system of claim 7, wherein the divalent metal ion is Mg²⁺ or Mn²⁺.

9. The coupled enzyme system of claim 1, wherein the divalent metal ion Mg²⁺ or Mn²⁺ to polyP ratio is 1:1.

10. The coupled enzyme system of claim 1, further comprise a cosolvent and a surfactant.

11. The coupled enzyme system of claim 1, wherein the surfactant is TWEEN 20 or TWEEN 80.
